(19) United States
(12) Patent Application Publication
(10) Pub. No.: US 2018/0037197 A1
(43) Pub. Date: Feb. 8, 2018

(54) METHOD OF CLEANING A MULTIDIRECTIONAL WHEEL

(71) Applicant: FRESENIUS MEDICAL CARE DEUTSCHLAND GMBH, Bad Homburg (DE)

(72) Inventors: Matthias BRANDL, Bad Koenigshofen (DE); Thomas FAULHABER, Bergrheinfeld (DE); Pavel MELINIC, Giessen (DE)

(73) Assignee: FRESENIUS MEDICAL CARE DEUTSCHLAND GMBH, Bad Homburg (DE)

(21) Appl. No.: 15/555,095

(22) PCT Filed: Feb. 29, 2016
(86) PCT No.: PCT/EP2016/000351

§ 371 Date: Sep. 1, 2017

(30) Foreign Application Priority Data
Mar. 2, 2015 (DE) 10 2015 002 531.7

(51) Int. Cl. B60S 3/04; B60B 19/00; A61L 2/18

(52) U.S. Cl. CPC B60S 3/042; A61L 2/18; B60B 19/003; A61L 2202/15; A61L 2202/17; B60B 2200/26

(57) ABSTRACT

The present invention relates to a method of cleaning a multidirectional wheel, wherein the wheel has a wheel body which is rotatable about an axis and has a plurality of rotating bodies which are located at the outer periphery of the wheel body and which form the running surface of the wheel, wherein the wheel body has at least one hollow space which has at least one inlet opening and at least one outlet opening for a flushing fluid and wherein the method comprises the introduction of a flushing fluid through the inlet opening into the at least one hollow space of the wheel body and the washing around of the rotating body by the flushing fluid after the discharge of the flushing fluid from the outlet opening.

The present invention relates to a method of cleaning a multidirectional wheel, wherein the wheel has a wheel body which is rotatable about an axis and has a plurality of rotating bodies which are located at the periphery of the wheel body and which form the running surface of the wheel.

It is known from the prior art e.g. to provide medical devices such as dialysis devices with casters to ensure their movability. Swivel casters or trestle casters are used as the casters, for example. In this kind of construction, the casters are generally fixedly connected to the machine frame. If such a caster is to be cleaned after contamination, e.g. by dirt on the floor, etc., this is sometimes only possible with difficulty.

The quality of such a cleaning of the caster substantially depends on the thoroughness of the person who carries out the cleaning work.

If the casters are largely or completely covered by covers, etc., the cleaning of the casters becomes correspondingly more difficult.

Furthermore, so-called multidirectional wheels are known from the prior art, i.e. wheels with which a movement in more than one direction is possible.

Such a multidirectional wheel is known, for example, from WO 2011/113562 A2.

Such a known multidirectional wheel comprises a wheel body as well as a plurality of rotating bodies, in particular rollers, which are located at the outer periphery of the wheel body. Provision can be made in this respect that the longitudinal axis of the rollers extends either the rotational plane of the wheel body or at an angle thereto.

Reference is furthermore made to DE 193 99 32 A1, AU 198 547 668, WO 00/12327 A1 and U.S. Pat. No. 7,641,288 which likewise disclose such wheel constructions and/or multidirectional wheels.

It is the underlying object of the present invention to provide a method by means of which a multidirectional wheel can be cleaned thoroughly in a comparatively simple manner.

This object is achieved by a method having the features of claim 1. Provision is accordingly made that the wheel body has at least one hollow space which has at least one inlet opening and at least one outlet opening for a flushing fluid. The method comprises the step of introducing the flushing fluid, such as a disinfectant, etc., through the inlet opening into the at least one hollow space of the wheel body and the washing around of the rotating bodies by the flushing fluid after the discharge of the flushing fluid from the at least one outlet opening.

The rotating body can, for example, be a sphere or a roller. If it is a roller, provision can be made that its longitudinal axis extends in the rotational plane of the wheel body or also at an angle thereto.

Provision is thus made in accordance with the invention that a flushing fluid is introduced into the wheel body and this flushing fluid then flows through the named hollow space of the wheel body and flows around the rotating bodies so that they can be cleaned and/or disinfected.

Provision is preferably made that the wheel body has a plurality of hollow spaces and that the method comprises the step of the preferably simultaneous introduction of the flushing fluid into the plurality of hollow spaces. It is possible in this manner to introduce flushing fluid into the hollow space or into the hollow spaces of the wheel body at several points and preferably simultaneously.

It is, for example, conceivable that the flushing fluid flows through the hollow space of the wheel body from the inside to the outside, i.e. in the radial direction or also obliquely thereto.

Provision is made in a further embodiment that the method comprises the placing or inserting of a flushing adapter on or into the wheel body, with the flushing fluid flowing out of the flushing adapter into the at least one hollow space of the wheel body. It is thus conceivable that a flushing adapter is, for example, placed onto the at least one inlet opening of the wheel body and the flushing fluid then flows through the hollow space of the wheel body and flows around the rotating bodies after the discharge through the outlet opening(s).

This flushing adapter has a single opening from which the flushing fluid is discharged or also preferably has a plurality of outlets through which the flushing fluid is discharged.

The method is preferably carried out such that the flushing fluid flows through the hollow space of the wheel body from the inside to the outside. It is thus conceivable, for example, that the wheel body has a central inlet opening and the flushing fluid flows from there radially or toward the radial direction obliquely outwardly, i.e. in the direction of the rotating bodies.

Provision can alternatively or additionally be made that the hollow space is configured such that a flowing through takes place in the peripheral direction or obliquely thereto. It is thus conceivable that the flushing fluid flows through the hollow space of the wheel body at least sectionally in the peripheral direction of the wheel body.

A combination of the two aforesaid flow directions (direction components in the tangential and radial direction) is likewise conceivable.

Provision is made in a further embodiment of the invention that the method comprises the step of removing the wheel from a device, in particular from a dialysis device, and that the flushing fluid is introduced into the wheel removed from the device. It is thus conceivable, for example, that the caster, i.e. the wheel with e.g. a quick-release fastener, can be removed from the device by a user. A foldable support can then e.g. take over the support function of the caster depending on the weight of the device. With lighter devices or racks, the total rack can also be placed on its side.

After the removal of the wheel, the flushing through of the wheel can then be carried out, preferably using the above-named flushing adapter.

Provision is made in a preferred embodiment of the invention that the flushing fluid is introduced into the hollow space or into the hollow spaces of the wheel body at a plurality of different positions. A particularly thorough cleaning of the hollow spaces and also of the rotating bodies is possible in this manner.

This plurality of positions can be arranged spaced apart from one another in the peripheral direction and/or in the radial direction of the wheel body.

The present invention further relates to a multidirectional wheel, wherein the wheel has a wheel body which is rotatable about an axis and has a plurality of rotating bodies which are located at the periphery of the wheel body and which form the running surface of the wheel.

Provision is made in accordance with the invention that the wheel body has at least one hollow space which has at least one inlet opening and at least one outlet opening for a flushing fluid and that the at least one outlet opening for the flushing fluid is arranged in the region of the rotating bodies.

Provision is made in a further embodiment of the invention that a plurality of hollow spaces, in particular a plurality of channels, are provided in the wheel body which have at least one outlet opening for the flushing fluid in the region of the rotating bodies.

Provision can furthermore be made that exactly one outlet opening per rotating body is provided or that a plurality of outlet openings are provided per rotating body.

A particularly thorough cleaning, in particular of roller-like rotating bodies, can be achieved when the outlet opening is of slit form and preferably extends along the rotating body or along its longitudinal axis.

It is also conceivable that a plurality of outlet openings are provided which preferably extend along the longitudinal axis of the rotating body. It is possible in this manner to apply the flushing fluid not only at one point, but distributed at a plurality of positions to the surface of the rotating body such as a roller or the like.

The present invention furthermore relates to an apparatus comprising at least one source for a flushing fluid, at least one flushing adapter which communicates with the source and which has at least one inlet opening, which communicates with the source, and at least one outlet opening for introducing the flushing fluid into the wheel. The apparatus furthermore has at least one wheel in accordance with the present invention.

Provision is preferably made that the flushing adapter has at least one tubular section, preferably a plurality of tubular sections, which communicate with the source for a flushing fluid. These tubular sections can be introduced into corresponding cut-outs of the wheel body and the flushing process can then be initiated.

Provision is made in a further embodiment of the invention that the outlet openings of the flushing adapter are spaced apart from one another in the peripheral direction and/or in the radial direction of the flushing adapter.

It is thus possible to introduce flushing fluid at a plurality of different positions of the wheel body.

Further details and advantages of the invention will be explained in more detail with reference to an embodiment described in the drawing.

There are shown:

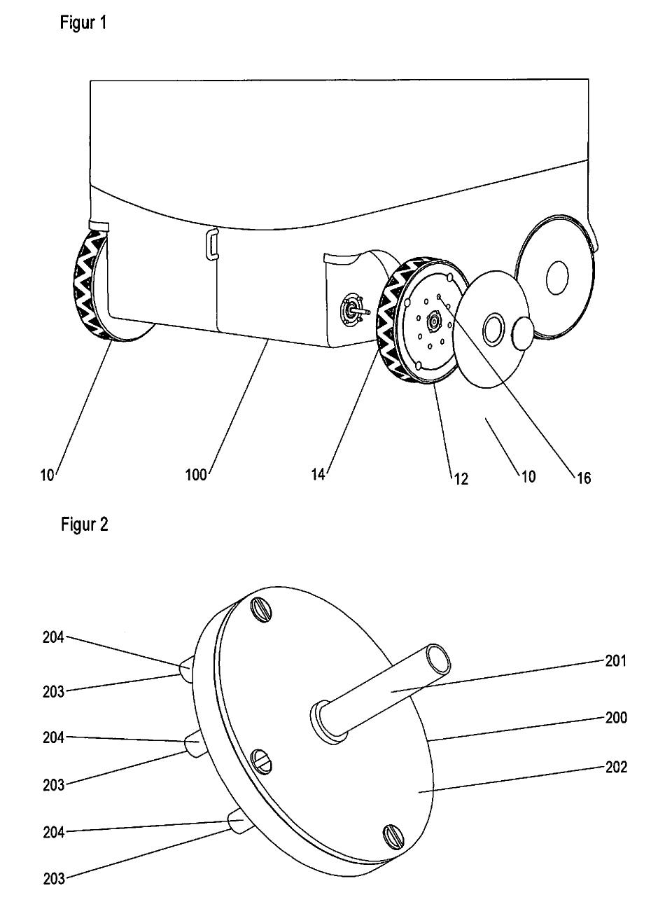

FIG. 1: a perspective view of the lower region of a dialysis device with a removed multidirectional wheel in accordance with the invention; and

FIG. 2: a perspective view of the flushing adapter.

The housing of a medical device such as a dialysis device is marked by the reference numeral 100 in FIG. 1.

The housing 100 is provided with four casters, with at least two rollers being formed by multidirectional wheels in accordance with the present invention.

As can be seen from FIG. 1, these wheels 10 comprise a wheel body 12 and a plurality of rotating bodies 14 which are configured in the form of rollers and extend along the outer surface of the wheel body 12.

As can be seen from FIG. 1, the longitudinal axes or the rotational axes of the rollers 14 do not extend in the rotational plane, i.e. not in the longitudinal sectional plane of the wheel body 12, but at an angle thereto.

Due to the presence of the rotating bodies 14, the wheel 10 cannot only be moved in the direction of the rotational plane, i.e. the longitudinal sectional plane of the wheel body 12, but also at an angle thereto, in accordance with the arrangement of the rotating bodies 14.

As can further be seen from FIG. 1, a plurality of inlet openings for a flushing fluid are located offset from one another in the peripheral direction in the wheel body. These inlet openings are marked by the reference numeral 16 and are arranged in circular form.

Apart from this arrangement, it is conceivable to arrange these inlet openings in a different manner or also to use more or fewer than the shown eight inlet openings 16 per wheel body 12.

FIG. 2 shows a flushing adapter 200 in a perspective view obliquely from above. This flushing adapter 200 has a base body 202 which has the shape of a circular disk in the embodiment shown here. The flushing adapter 200 furthermore has a plurality of outlet openings 203 which extend in the end regions of stub-shaped tubular sections 204.

Reference numeral 201 marks a central inlet opening which is, for example, in communication with the shown hose and through which a flushing fluid enters into the inner region of the flushing adapter 200.

After the entry into the inner region or hollow space of the flushing adapter 200, the flushing fluid is distributed over the tube lines 04 and is discharged from the flushing adapter through the outlet openings 203.

After the user has removed the wheel 10 from the axle of the device shown in FIG. 1 by, for example, releasing a quick-release fastener, the wheel 10 is connected to the flushing adapter 200 in that the tubular stubs 204 are introduced into the openings 16 of the wheel 10.

The flushing fluid is subsequently introduced from a source not shown into the flushing adapter 200 and from this into the inner region of the wheel or of the wheel body 12.

Channels or other hollow spaces extend outwardly from there through the inner region of the wheel body 12, i.e. toward the peripheral surface, at which the rotating bodies 14 are located. The flushing fluid is thus discharged in the region of the peripheral surface of the wheel body 12 at which the rotating bodies 14 are also arranged.

It is conceivable that the flushing fluid is discharged at a plurality of positions of the wheel body 12 distributed in the peripheral direction. It is preferred for at least one outlet opening to be provided for each rotating body 14 so that it is ensured that each rotating body 14 is acted on by a flushing fluid and preferably by a disinfecting fluid.

The hollow spaces or channels are preferably configured such that the fluid flows through the complete wheel body from the inside to the outside and removes dirt and germs from all individual casters or rotating bodies.

It is possible in this manner to achieve a cleaning or disinfecting of the wheel body itself and also of all rotating bodies which are located on the outer surface of the wheel body despite the comparatively complex design of the multidirectional wheel shown.

1. A method of cleaning a multidirectional wheel, wherein the wheel has a wheel body which is rotatable about an axis and has a plurality of rotating bodies which are located at the outer periphery of the wheel body and which form the running surface of the wheel, characterized in that the wheel body has at least one hollow space which has at least one inlet opening and at least one outlet opening for a flushing fluid; and in that the method comprises the introduction of a flushing fluid through the inlet opening into the at least one hollow space of the wheel body and the washing around of the rotating body by the flushing fluid after the discharge of the flushing fluid from the outlet opening.

2. A method in accordance with claim 1, characterized in that the wheel body has a plurality of hollow spaces; and in that the method comprises the step of the preferably simultaneous introduction of the flushing fluid into the plurality of hollow spaces.

3. A method in accordance with claim 1, characterized in that the method comprises the placing or inserting of a flushing adapter on or into the wheel body, with the flushing fluid flowing out of the flushing adapter into the at least one hollow space of the wheel body.

4. A method in accordance with claim 1, characterized in that the flushing fluid flows through the hollow space of the wheel body from the inside to the outside.

5. A method in accordance with claim 1, characterized in that the flushing fluid flows through the hollow space of the wheel body in the peripheral direction of the wheel body.

6. A method in accordance with claim 1, characterized in that the method comprises the step of removing the wheel from a device, in particular from a dialysis device; and in that the flushing fluid is introduced into the wheel removed from the device.

7. A method in accordance with claim 1, characterized in that the flushing fluid is introduced into the hollow space or spaces of the wheel body at a plurality of different positions.

8. A method in accordance with claim 7, characterized in that the plurality of positions are spaced apart from one another in the peripheral direction and/or in the radial direction of the wheel body.

9. A multidirectional wheel, wherein the wheel has a wheel body which is rotatable about an axis and has a plurality of rotating bodies which are located at the outer periphery of the wheel body and which form the running surface of the wheel, characterized in that the wheel body has at least one hollow space which has at least one inlet opening and at least one outlet opening for a flushing fluid; and in that the at least one outlet opening for the flushing fluid is arranged in the region of the rotating bodies.

10. A multidirectional wheel in accordance with claim 9, characterized in that a plurality of hollow spaces, in particular a plurality of channels, are provided which have the at least one outlet opening for the flushing fluid in the region of the rotating bodies.

11. A multidirectional wheel in accordance with claim 9, characterized in that exactly one outlet opening is provided per rotating body; or in that a plurality of outlet openings are provided per rotating body.

12. A multidirectional wheel in accordance with claim 9, characterized in that the outlet opening is in the form of a slit and preferably extends along the rotating body; or in that a plurality of outlet openings are provided which are preferably distributed along the rotating body.

13. An apparatus comprising at least one source for a flushing fluid, at least one flushing adapter which communicates with the source and which has at least one inlet opening, which communicates with the source, and at least one outlet opening for introducing the flushing fluid into the wheel, and comprising at least one wheel in accordance with claim 9.

14. An apparatus in accordance with claim 13, characterized in that the flushing adapter has at least one tubular section, preferably a plurality of tubular sections, which communicate with the source for a flushing fluid.

15. An apparatus in accordance with claim 13, characterized in that the outlet openings are spaced apart from one another in the peripheral direction and/or in the radial direction of the flushing adapter.
